SEQUENCE LISTING (27 sequences)

SEQ ID NO: 1 - protein, 8 aa, synthetic construct
GFTFRDYA

SEQ ID NO: 2 - protein, 8 aa, synthetic construct
ISGSGGRT

SEQ ID NO: 3 - protein, 18 aa, synthetic construct
AKDRLSITIRPRYYGLEV

SEQ ID NO: 4 - protein, 11 aa, synthetic construct
QSLLYSIGYNY

SEQ ID NO: 5 - protein, 9 aa, synthetic construct
MQALQTPYT

SEQ ID NO: 6 - protein, 125 aa, synthetic construct
EVQLVESGGGLEQPGGSLRLSCAGSGFTFRDYAMTWVRQAPGKGLEWVSSISGSGGRTYYADSVKGRFTISRDNSKNTLYLQMNSLRAEDTAVYYCAKDRLSITIRPRYYGLEVWGQGTTVTVSS

SEQ ID NO: 7 - protein, 112 aa, synthetic construct [sequence omitted]

SEQ ID NO: 8 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 107 aa, synthetic construct [sequence omitted]

SEQ ID NO: 10 - protein, 8 aa, synthetic construct
ISGSGGNT

SEQ ID NO: 11 - protein, 18 aa, synthetic construct
AKDRLSITIRPRYYGLWV

SEQ ID NO: 12 - protein, 125 aa, synthetic construct
EVQLVESGGGLVQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLWVWGQGTTVTVSS

SEQ ID NO: 13 - protein, 112 aa, synthetic construct
DIVMTQSPLSLPVTPGEPASISCRSSQSLLYSIGYNYLDWYLQKSGQSPKLLIYLGSNRASGVPDRFSGSGSGTDFTLKISRVEAEDVGFYYCMQALQTPYTFGQGTKLEIK

SEQ ID NO: 14 - protein, 329 aa, synthetic construct; identical to SEQ ID NO: 8

SEQ ID NO: 15 - protein, 18 aa, synthetic construct
AKDRLSITIRPRYYGLDV

SEQ ID NO: 16 - protein, 125 aa, synthetic construct [sequence omitted]

SEQ ID NO: 17 - protein, 5 aa, synthetic construct
AYSVN

SEQ ID NO: 18 - protein, 16 aa, synthetic construct
MIWGDGKIVYNSALKS

SEQ ID NO: 19 - protein, 10 aa, synthetic construct
DGYYPYAMDN

SEQ ID NO: 20 - protein, 15 aa, synthetic construct
RASKSVDSYGNSFMH

SEQ ID NO: 21 - protein, 7 aa, synthetic construct
LASNLES

SEQ ID NO: 22 - protein, 9 aa, synthetic construct
QQNNEDPRT

SEQ ID NO: 23 - protein, 118 aa, synthetic construct [sequence omitted]

SEQ ID NO: 24 - protein, 111 aa, synthetic construct [sequence omitted]

SEQ ID NO: 25 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 26 - protein, 825 aa, Macaca fascicularis
MGWLCSGLLFPVSCLVLLQVASSGSMKVLQEPACVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQRLLWGVSAACVFILFFCLSCYFSVTKIKKEWWDQIPNPARSHLVAIIIQDAQESQWEKRSRGQEAAKCPYWKNCLTKLLPCFLEHNMKRDEDPHKAVKDLPFRGSGKSAWCPVEISKTVLWPESISVVRCVELFEAPVECKEEEEVEEEKGSFCTSSESNRDDFQEGREGIVARLTESLFLDLLGGENGGFFQQDMGESCLLPPLGSTSAHVPWDEFPSAGPKEVPPWGKEQPLHQEPSPPASPTQSPDNPTCTEMPLVISSNPAYRSFSNSLSQSPCPRELGPDPLLARHLEEVDPEMPCAPQLSEPTTVAPAEPETWEQILRRNVLQHGAAAAPASAPTSGYREFVHAVQQGGIQASAVAGLGPPGEAGYKAFSSLLASSAVSPGECGFGASSGEEGYKPFQDLTPGCPGDPAPVPVPLFTFGLDREPPHSPQSSHLPSNSPEHLALEPGEKVEDMQKPPLPPEQATDPLGDNLGSGIVYSALTCHLCGHLKQCHGQEDGGQAPVVASPCCGCCCGDRSSPPTTPLRAPDPSLGGVPLEASLCLASLAPSGISEKSKSSLSFHPAPGSAQSSSQTPQIVNFVSVGPTCMRVS

SEQ ID NO: 27 - protein, 825 aa, Macaca mulatta
MGWLCSGLLFPVSCLVLLQVASSGSMKVLQEPTCVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDLADSRIHNVTYLKPTLRIPASTLKSGISYRARVRAWAQHYNTTWSEWSPSTKWYNSYREPFEQRLLWGVSAACVFILFFCLSCYFSVTKIKKEWWDQIPNPARSHLVAIIIQDAQESQWEKRSRGQEAAKCPYWKNCLTKLLPCFLEHNMKRDEDPHKAVKDLPFRGSGKSAWCPVEISKTVLWPESISVVRCVELFEAPVECKEEEEVEEEKGSFCTSSESNRDDFQEGREGIVARLTESLFLDLLGGENGGFFQQDMGESCLLPPLGSTSAHVPWDEFPSAGPKEVPPWGKEQPLHQEPSPPASPTQSPDNPTCTEMPLVISSNPAYRSFSNSLSQSPCPRELGPDPLLARHLEEVDPEMPCDPQLSEPTTVAPAEPETWEQILRRNVLQHGAAAAPASAPTSGYREFVHAVQQGGIQASAVAGLGPPGEAGYKAFSSLLASSAVSPGECGFGASSGEEGYKPFQDLTPGCPGDPAPVPVPLFTFGLDREPPHSPQSSHLPSNSPEHLALEPGEKVEDMQKPPLPPEQATDPLGDNLGSGIVYSALTCHLCGHLKQCHGQEDGGQAPVVASPCCGCCCGDRSSPPTTPLRAPDPSLGGVPLEASLCPASLAPSGISEKSKSSLSFHPAPGSAQSSSQTPQIVNFVSVGPTCMRVS

(19) United States
(12) Patent Application Publication
(10) Pub. No.: US 2026/0317433 A1
(43) Pub. Date: Oct. 15, 2026

(54) ANTI-INTERLEUKIN-4 RECEPTOR ALPHA ANTIBODIES AND USES THEREOF

(71) Applicant: Vial Health Technology Inc., San Francisco, CA (US)

(72) Inventors: Ritu Ratish Randhawa, Hamilton (CA); Preeti Featherston Sareen, Vernon Hills, IL (US); Sarah Jane Wise, Chapel Hills, NC (US)

(21) Appl. No.: 19/378,005

(22) Filed: Nov. 3, 2025

Related U.S. Application Data
(60) Provisional application No. 63/715,330, filed on Nov. 1, 2024.

(51) Int. Cl. C07K 16/28; A61K 47/68

(52) U.S. Cl. CPC C07K 16/2866; A61K 47/6849; C07K 2317/24; C07K 2317/31; C07K 2317/52; C07K 2317/565; C07K 2317/71; C07K 2317/73

(57) ABSTRACT

Provided herein are antibody molecules that bind specifically to IL-4Rα and antigen-binding portions thereof and related compositions, nucleic acid molecules, vectors and host cells. Also provided herein are medical uses of such antibody molecules.

### CROSS-REFERENCE TO RELATED APPLICATIONS

This application claims the benefit of U.S. Provisional Application No. 63/715,330, filed Nov. 1, 2024, the content of which is herein incorporated by reference in its entirety.

### REFERENCE TO AN ELECTRONIC SEQUENCE LISTING

The contents of the electronic sequence listing (VHT_002_00US_SeqList_ST26.xml; Size: 28,199 bytes; Date of Creation: Nov. 3, 2025) are herein incorporated by reference in their entirety.

### TECHNICAL FIELD

The present disclosure relates to therapeutic antibody molecules and medical uses thereof.

### BACKGROUND

The IL-4 receptor (IL-4R) is a heterodimeric structure composed of a common subunit, IL-4Rα that pairs with a γ common (γc) chain to form IL-4R type I, or with IL-13 receptor α (IL-13Rα) to form IL-4R type II. The type-2 cytokines IL-4 and IL-13 interact with the IL-4R receptor: IL-4R type I binds only IL-4, whereas IL-4R type II binds both IL-4 and IL-13. Disruption of signalling through IL-4R has been proposed as a target for treatment or prevention of a variety of allergic diseases including atopic dermatitis. There is a need for therapeutics that target such immune-mediated diseases.

### SUMMARY

Provided herein is an anti-IL4 receptor alpha (IL-4Rα) antibody or an antigen-binding portion thereof, wherein the antibody or antigen-binding portion comprises a heavy chain variable (VH) region and a light chain variable (VL) region wherein: (a) the VH region amino acid sequence comprises a heavy chain complementarity determining region 1 (HCDR1) comprising the amino acid sequence of SEQ ID NO: 1, a heavy chain complementarity determining region 2 (HCDR2) comprising the amino acid sequence of SEQ ID NO: 2 and a heavy chain complementarity determining region 3 (HCDR3) comprising the amino acid sequence of SEQ ID NO: 3; and the VL region amino acid sequence comprises a light chain complementarity determining region 1 (LCDR1) comprising the amino acid sequence of SEQ ID NO: 4, a light chain complementarity determining region 2 (LCDR2) comprising the amino acid sequence of LGS and a light chain complementarity determining region 1 (LCDR3) comprising the amino acid sequence of SEQ ID NO: 5; or (b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5.

Provided herein is an antibody or antigen-binding portion, wherein: (a) the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 6 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 7; or (b) the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 12 and the VL region amino acid sequence has at least 90%, %, at least 91%, at least 92%, at least 93%, at least 94%, at least 95%, at least 96%, at least 97%, at least 98%, or at least 99% sequence identity to the amino acid sequence of SEQ ID NO: 13.

Provided herein is an antibody or antigen-binding portion, wherein: (a) the VH region comprises the amino acid sequence of SEQ ID NO: 6 and the VL region comprises the amino acid sequence of SEQ ID NO: 7; or (b) the VH region comprises the amino acid sequence of SEQ ID NO: 12 and the VL region comprises the amino acid sequence of SEQ ID NO: 13.

In some embodiments, the antibody or antigen-binding portion is humanized or chimeric.

In some embodiments, the VH region, the VL region, or both the VH and the VL region comprise one or more human framework region amino acid sequences.

In some embodiments, the antibody or antigen-binding portion comprises an immunoglobulin constant region.

In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region is a human IgG1 or IgG4 constant region comprising the amino acid substitutions L234A and L235A, wherein numbering is according to the EU index as in Kabat. In some embodiments, the immunoglobulin constant region is a human IgG1 or IgG4 constant region comprising the amino acid substitutions M252Y, S254T, and T256E, wherein numbering is according to the EU index as in Kabat. In some embodiments, the immunoglobulin constant region comprises (i) any one of SEQ ID NOs: 8 or 14, and (ii) SEQ ID NO: 9.

In some embodiments, the antibody has reduced affinity for FcRn compared to dupilumab.

In some embodiments, the antibody or antigen-binding portion is an Fab, an Fab′, an F(ab′)2, an Fv, an scFv, a maxibody, a minibody, a diabody, a triabody, a tetrabody, or a bis-scFv. In some embodiments, the antibody or antigen-binding portion is bispecific. In some embodiments, the antibody or antigen-binding portion is linked to a therapeutic agent.

Presented herein is a pharmaceutical composition comprising any antibody or antigen-binding portion or immunoconjugate described herein, and a pharmaceutically acceptable carrier, diluent or excipient.

Presented herein is a nucleic acid molecule encoding both the VH and the VL region amino acid sequences of any antibody or antigen-binding portion described herein. Presented herein is an expression vector comprising any nucleic acid molecule described herein. Presented herein is a recombinant host cell comprising any nucleic acid molecule described herein. Presented herein is recombinant host cell comprising a first nucleic acid molecule encoding the VH region amino acid sequence of the antibody or antigen-binding portion described herein, and a second nucleic acid molecule encoding the VL region amino acid sequence of said antibody or antigen-binding portion.

Presented herein is a method of producing an anti-IL-4Rα antibody or an antigen-binding portion thereof described herein, the method comprising: (i) culturing the recombinant host cell of claim described herein under conditions whereby the nucleic acid molecule is expressed, thereby producing the antibody or antigen-binding portion; and (ii) isolating the antibody or antigen-binding portion from the host cell or culture.

Presented herein is a method for treating or preventing a disease or condition in a subject, comprising administering to the subject a therapeutically effective amount of an antibody or antigen-binding portion, immunoconjugate, or pharmaceutical composition described herein.

Presented herein is a method for reducing or improving at least one symptom of a disease or condition in a subject, comprising administering to the subject a therapeutically effective amount of an antibody or antigen-binding portion, immunoconjugate, or pharmaceutical composition described herein. In some embodiments, the method results in predicted improvement of one or more pharmacokinetic properties. In some embodiments, the method results in a predicted improvement in immunogenicity. In some embodiments, the method results in reduced IL-4/IL-13 signalling. In some embodiments, the disease or condition is associated with increased IL-4/IL-13 production.

In some embodiments, the disease or condition is atopic dermatitis. In some embodiments, the disease or condition is chronic obstructive pulmonary disorder (COPD), asthma, prurigo nodularis, or chronic rhinosinusitis with nasal polyps (CRSwNP).

Presented herein is a method for reducing IL-4/IL-13 signalling, wherein the method comprises contacting a cell with an antibody or antigen-binding portion, or immunoconjugate described herein. In some embodiments, the method results in reduced proliferation of the cell. In some embodiments, the method results in reduced production of a cytokine, preferably TARC.

Presented herein is an antibody or antigen-binding portion, immunoconjugate, or pharmaceutical composition described herein, for use as a medicament.

### BRIEF DESCRIPTION OF THE DRAWINGS

FIG. 1 provides a diagram of exemplary mutations in the immunoglobulin constant region of antibodies described herein.

### DETAILED DESCRIPTION

Provided herein are anti-IL-4Rα antibodies and therapeutic uses of such antibodies. The antibodies disclosed herein have favorable pharmacokinetics, low immunogenicity, and high efficacy.

The IL-4 receptor (IL-4R) is a heterodimeric structure. The common subunit IL-4Rα (UniProt ID: P24394) can pair with a γ common (γc) chain to form IL-4R type I, or it can pair with the IL-13 receptor (IL-13Rα1) to form IL-4R type II. Upon binding of IL-4 to IL-4Rα, the IL-4/IL-4Rα-complex interacts with either γc or IL-13Rα1. The expression of γc and IL-13Rα1 varies among cells: hematopoietic cells generally express relatively high amounts of γc and little IL-13 Rα1, whereas non-hematopoietic cells generally express relatively high levels of IL-13Rα1 and little or no γc. Disruption of signalling through IL-4R has been proposed as a target for treatment or prevention of a variety of allergic diseases including atopic dermatitis. There is a need for therapeutics that target such immune-mediated diseases.

IL-4R signaling can involve one or more pathways including JAK-STAT mediated phosphorylation of STAT6, IRS-mediated activation of PI3K/AKT/mTOR, and ERK1/2, p38 MAPK, and SRC pathways. Signaling through IL-4R is central to the pathology associated with allergic diseases and has been implicated in diseases and conditions characterized by allergic inflammation such as atopic dermatitis, asthma, and food allergies. Disruption of IL-4R signaling has been proposed as a target for treatment of such diseases.

Dupilumab, a human monoclonal antibody that binds IL-4Rα, has received FDA approval for treatment of dermatitis, asthma, chronic rhinosinusitis with nasal polyposis, eosinophilic esophagitis, and prurigo nodularis. However, this antibody has a number of limitations including pharmacokinetic properties that require administration every two weeks. Another therapeutic targeting this pathway, lebrikizumab (an anti-interleukin-13 antibody), has shown immunogenicity concerns, which could limit its long-term efficacy and safety in patients. These limitations demonstrate the need for development of antibodies with improved pharmacokinetics, reduced immunogenicity, and equipotent or superior efficacy.

In contrast, the anti-IL-4Rα antibodies provided herein demonstrate advantages as described herein that make them useful for treatment of diseases and conditions characterized by allergic inflammation.

### Antibodies

Provided herein are antibodies and antigen-binding portions thereof that specifically bind IL-4Rα. The anti-IL-4Rα antibodies provided herein have several advantages over the anti-IL-4Rα antibody dupilumab (disclosed, e.g., in U.S. Pat. No. 7,608,693). The anti-IL-4Rα antibodies provided herein have been engineered to have improved potency in blocking IL-4 and/or IL-13 signaling through IL-4Rα. Critically, these antibodies also have improved pharmacokinetic properties (e.g., longer half-life) compared to dupilumab.

Antibodies and antigen-binding portions disclosed herein specifically bind human IL-4Rα. In some embodiments, antibodies and antigen-binding portions may cross-react with IL-4Rα from species other than human, for example, cynomolgus monkey (Macaca fascicularis) IL-4Rα and/or rhesus monkey (Macaca mulatta) IL-4Rα. In some embodiments, an antibody may be specific for only human IL-4Rα and may exhibit no non-human cross-reactivity. Exemplary amino acid sequences of human, cynomolgus and rhesus IL-4Rα are provided in Table 4.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable region (abbreviated herein as VH region) and a heavy chain constant region. The heavy chain constant region comprises three domains, CH1, CH2 and CH3. Each light chain comprises a light chain variable region (abbreviated herein as VL region) and a light chain constant region. The light chain constant region comprises one domain, CL. The VH and VL regions can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4. The CDR definition used in the present application is the Kabat definition (Kabat et al., Sequences of Proteins of Immunological Interest, 5th ed. Bethesda, MD: Public Health Service, National Institutes of Health (1991)).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgAQ1 or IgA2) or subclass. IgG, IgD, and IgE antibodies generally contain two identical heavy chains and two identical light chains and two antigen combining domains, each composed of a VH) and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., IL-4Rα). It has been shown that the antigen-binding function of an antibody can be performed by portions or fragments of a full-length antibody. Examples of binding portions encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′) 2 fragments. Fab′ is derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody portion”. An antibody portion generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen is IL-4Rα and the second antigen is not IL-4Rα. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-IL-4Rα antigen-binding portion disclosed herein is an Fab, an Fab′, an F(ab′)₂, an Fv, an scFv, a maxibody, a minibody, a diabody, a triabody, a tetrabody, or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind IL-4Rα when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art. One method for determining the K_{d} of an antibody is by using surface plasmon resonance (SPR), typically using a biosensor system such as a Biacore® system.

In some embodiments, an anti-IL-4Rα antibody or antigen-binding portion provided herein is monovalent or bivalent and comprises a single or double chain. Functionally, the binding affinity of an antibody or antigen-binding portion may be within the range of 10⁻⁵M to 10⁻¹² M. For example, the binding affinity of an antibody or antigen-binding portion is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷M or from 10⁻⁵ M to 10⁻⁶ M.

In some embodiments, an anti-IL-4Rα antibody or an antigen-binding portion thereof does not comprise one or more of the dupilumab amino acid sequences provided in Table 3. In some embodiments, an anti-IL-4Rα antibody or an antigen-binding portion thereof does not comprise an HCDR1 of SEQ ID NO: 1, HCDR2 of SEQ ID NO: 10, HCDR3 of SEQ ID NO: 15, LCDR1 of SEQ ID NO: 4, LCDR2 of LGS and LCDR3 of SEQ ID NO: 5. Table 3 provides the amino acid sequences of the dupilumab anti-IL-4Rα antibody variable regions with highlighted CDRs as defined herein (“Kabat” scheme).

The antibodies disclosed herein are anti-IL-4Rα antagonist antibodies. As used herein, an “antagonist” or an “anti-IL-4Rα antagonist antibody” (interchangeably termed “anti-IL-4Rα antibody”) refers to an antibody which is able to bind to IL-4Rα and inhibit IL-4Rα biological activity and/or downstream pathway(s) mediated by IL-4Rα signalling. An anti-IL-4Rα antagonist antibody encompasses antibodies that can block, antagonize, suppress or reduce (including significantly) IL-4Rα biological activity, including downstream pathways mediated by IL-4Rα signalling, such as receptor binding and/or elicitation of a cellular response to IL-4Rα. For the purposes of the present disclosure, it will be explicitly understood that the term “anti-IL-4Rα antagonist antibody” encompasses all the terms, titles, and functional states and characteristics whereby IL-4Rα itself, and IL-4Rα biological activity (including but not limited to its ability to suppress the activation of anti-tumor cell activity of T cells), or the consequences of the activity or biological activity, are substantially nullified, decreased, or neutralized in a meaningful degree.

Further provided herein is an anti-IL-4Rα antibody or an antigen-binding portion thereof that comprises a combination of a VH region amino acid sequence and VL region amino acid sequence, and/or a combination of CDR amino acid sequences provided in Tables 1 and 2. In some embodiments, the VH region amino acid sequence and/or the VL region amino acid sequence comprises a signal sequence (also known as a signal peptide) at the amino-terminus.

Provided herein is an anti-IL-4Rα antibody or an antigen-binding portion thereof, wherein the antibody or antigen-binding portion comprises a heavy chain variable (VH) region and a light chain variable (VL) region wherein: (a) the VH region amino acid sequence comprises a HCDR1 comprising SEQ ID NO: 1, a HCDR2 comprising SEQ ID NO: 2 and a HCDR3 comprising SEQ ID NO: 3; and the VL region amino acid sequence comprises a LCDR1 comprising SEQ ID NO: 4, a LCDR2 comprising LGS and a LCDR3 comprising SEQ ID NO: 5; or (b) the VH region amino acid sequence comprises a HCDR1 comprising SEQ ID NO: 1, a HCDR2 comprising SEQ ID NO: 10 and a HCDR3 comprising SEQ ID NO: 11; and the VL region amino acid sequence comprises a LCDR1 comprising SEQ ID NO: 4, a LCDR2 comprising LGS and a LCDR3 comprising SEQ ID NO: 5.

Provided herein is an anti-IL-4Rα antibody or an antigen-binding portion thereof, wherein the antibody or antigen-binding portion comprises a VH region and a VL region. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 6. In some embodiments, the VH region comprises the amino acid sequence of SEQ ID NO: 6. In some embodiments, the VH region consists of the amino acid sequence of SEQ ID NO: 6. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 12. In some embodiments, the VH region comprises the amino acid sequence of SEQ ID NO: 12. In some embodiments, the VH region consists of the amino acid sequence of SEQ ID NO: 12.

In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 7. In some embodiments, the VL region comprises the amino acid sequence of SEQ ID NO: 7. In some embodiments, the VL region consists of the amino acid sequence of SEQ ID NO: 7. In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 13. In some embodiments, the VL region comprises the amino acid sequence of SEQ ID NO: 13. In some embodiments, the VL region consists of the amino acid sequence of SEQ ID NO: 13.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 6 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 7.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 12 and the VL region amino acid sequence has at least 90%, %, at least 91%, at least 92%, at least 93%, at least 94%, at least 95%, at least 96%, at least 97%, at least 98%, or at least 99% sequence identity to the amino acid sequence of SEQ ID NO: 13.

In some embodiments, the VH region comprises the amino acid sequence of SEQ ID NO: 6 and the VL region comprises the amino acid sequence of SEQ ID NO: 7. In some embodiments, the VH region comprises the amino acid sequence of SEQ ID NO: 12 and the VL region comprises the amino acid sequence of SEQ ID NO: 13. In some embodiments, the VH region consists of the amino acid sequence of SEQ ID NO: 6 and the VL region consists of the amino acid sequence of SEQ ID NO: 7. In some embodiments, the VH region consists of the amino acid sequence of SEQ ID NO: 12 and the VL region consists of the amino acid sequence of SEQ ID NO: 13.

Provided herein is an anti-IL-4Rα antibody or an antigen-binding portion thereof, wherein the antibody or antigen-binding portion comprises a VH region and a VL region, wherein (a) the VH region amino acid sequence comprises SEQ ID NO: 6; and the VL region amino acid sequence comprises SEQ ID NO: 7, with 1, 2 or 3 conservative amino acid substitutions in the VH region sequence, the VL region sequence, or both the VH region and the VL region sequences; or (b) the VH region amino acid sequence comprises SEQ ID NO: 12; and the VL region amino acid sequence comprises SEQ ID NO: 13, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

In some embodiments, an anti-IL-4Rα antibody or antigen-binding portion provided herein is monoclonal. The term “monoclonal antibody” (Mab) refers to an antibody, or antigen-binding portion thereof, that is derived from a single copy or clone, including for example any eukaryotic, prokaryotic, or phage clone, and not the method by which it is produced. Preferably, a monoclonal antibody exists in a homogeneous or substantially homogeneous population.

In some embodiments, the antibody or antigen-binding portion provided herein may be isolated.

In some embodiments, an anti-IL-4Rα antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1 or an IgG4 human constant region. Examples of chimeric antibodies and suitable techniques for their generation are provided in U.S. Pat. Nos. 4,816,567; 4,975,369; and 4,816,397, each of which is incorporated herein by reference in its entirety. In some embodiments, an anti-IL-4Rα antibody or an antigen-binding portion provided herein comprises: (a) a VH region amino acid sequence comprising SEQ ID NO: 6; a VL region amino acid sequence comprising SEQ ID NO: 7 and a human constant region; or (b) a VH region amino acid sequence comprising SEQ ID NO: 12, a VL region amino acid sequence comprising SEQ ID NO: 13 and a human constant region.

In some embodiments, an anti-IL-4Rα antibody or antigen-binding portion provided herein is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. An anti-IL-4Rα antibody molecule or antigen-binding portion thereof may comprise one or more human variable region framework scaffolds into which the CDRs have been inserted. In some embodiments, the VH region, the VL region, or both the VH region and the VL region of an anti-IL-4Rα antibody or antigen-binding portion provided herein comprise one or more human framework region amino acid sequences. In some embodiments, a humanized antibody comprises sequences that are entirely human except for the CDRs, which are the CDRs of antibody AB-IL-4Rα-02-0084 and AB-IL-4Rα-02-011. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and WO 90/07861, each of which is incorporated herein by reference in its entirety. When choosing FR to flank CDRs, for example when humanizing or optimizing an antibody, FRs from antibodies that contain CDR sequences in the same canonical class are preferred.

Also provided herein is an anti-IL-4Rα antibody or an antigen-binding portion thereof, wherein the antibody or antigen-binding portion comprises a VH region, a VL region and all human framework region sequences, wherein: (a) the VH region amino acid sequence comprises a HCDR1 of SEQ ID NO: 1, a HCDR2 of SEQ ID NO: 2 and a HCDR3 of SEQ ID NO: 3; and the VL region amino acid sequence comprises a LCDR1 of SEQ ID NO: 4, a LCDR2 of LGS and a LCDR3 of SEQ ID NO: 5; or (b) the VH region amino acid sequence comprises a HCDR1 of SEQ ID NO: 1, a HCDR2 of SEQ ID NO: 10 and a HCDR3 of SEQ ID NO: 11; and the VL region amino acid sequence comprises a LCDR1 of SEQ ID NO: 4, a LCDR2 of LGS and a LCDR3 of SEQ ID NO: 5.

Also provided herein is an anti-IL-4Rα antibody or an antigen-binding portion thereof, wherein the antibody or antigen-binding portion comprises a VH region, a VL region and one or more human framework region sequences, wherein: (a) the VH region amino acid sequence comprises a HCDR1 of SEQ ID NO: 1, a HCDR2 of SEQ ID NO: 2 and a HCDR3 of SEQ ID NO: 3; and the VL region amino acid sequence comprises a LCDR1 of SEQ ID NO: 4, a LCDR2 of LGS and a LCDR3 of SEQ ID NO: 5; or (b) the VH region amino acid sequence comprises a HCDR1 of SEQ ID NO: 1, a HCDR2 of SEQ ID NO: 10 and a HCDR3 of SEQ ID NO: 11; and the VL region amino acid sequence comprises a LCDR1 of SEQ ID NO: 4, a LCDR2 of LGS and a LCDR3 of SEQ ID NO: 5.

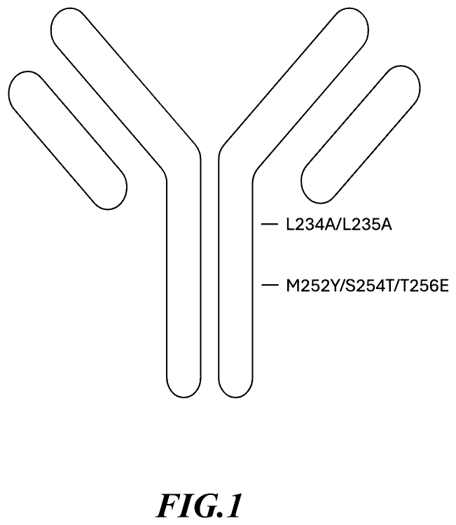

In some embodiments, an anti-IL-4Rα antibody or an antigen-binding portion thereof provided herein comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgAQ1 or IgA2. In some embodiments, the immunoglobulin constant region is IgG1 or IgG4. In some embodiments, the immunoglobulin constant region comprises one or more mutations to increase half-life. Non-limiting examples of immunoglobulin constant region mutations are shown in FIG. 1. In some embodiments, the immunoglobulin constant region is immunologically inert. In some embodiments, the immunoglobulin constant region comprises one or more mutations to reduce or prevent FcγR binding, antibody-dependent cell-mediated cytotoxicity activity, and/or complement-dependent cytotoxicity activity. In some embodiments, the immunoglobulin constant region is a wild-type human IgG1 constant region, a wild-type human IgG4 constant region, a human IgG1 constant region comprising the amino acid substitutions L234A and L235A, or a human IgG4 constant region comprising the amino acid substitutions L234A and L235A, wherein numbering is according to the EU index as in Kabat. In some embodiments, the immunoglobulin constant region is a human IgG1 constant region comprising the amino acid substitutions M252Y, S254T, and T256E, or a human IgG4 constant region comprising the amino acid substitutions M252Y, S254T, and T256E, wherein numbering is according to the EU index as in Kabat. In some embodiments, the immunoglobulin constant region is a human IgG1 constant region comprising the amino acid substitutions L234A, L235A, M252Y, S254T, and T256E, or a human IgG4 constant region comprising the amino acid substitutions L234A, L235A, M252Y, S254T, and T256E, wherein numbering is according to the EU index as in Kabat. In some embodiments, the immunoglobulin constant region is a human IgG1 constant region comprising one or more of the amino acid substitutions L234A, L235A, M252Y, S254T, and T256E, or a human IgG4 constant region comprising one or more of the amino acid substitutions L234A, L235A, M252Y, S254T, and T256E, wherein numbering is according to the EU index as in Kabat. In some embodiments, a position of an amino acid residue in a constant region of an immunoglobulin molecule is numbered according to the EU index as in Kabat (Ward et al., 1995 Therap. Immunol. 2:77-94).

In some embodiments, an anti-IL-4Rα antibody or an antigen-binding portion thereof provided herein may comprise an immunoglobulin light chain constant region that is a kappa light chain constant region or a lambda light chain constant region.

In some embodiments, an anti-IL-4Rα antibody or an antigen-binding portion thereof provided herein may comprise an immunoglobulin constant region comprising any one of the amino acid sequences in Table 5. The Fc region sequences in Table 5 begin at the CH1 domain. In some embodiments, an anti-IL-4Rα antibody or an antigen-binding portion thereof provided herein may comprise an immunoglobulin heavy chain constant region comprising any one of SEQ ID NOs: 8 and 14. In some embodiments, an anti-IL-4Rα antibody or an antigen-binding portion thereof provided herein may comprise an immunoglobulin light chain constant region comprising SEQ ID NO: 9. In some embodiments, an anti-IL-4Rα antibody or an antigen-binding portion thereof provided herein may comprise an immunoglobulin constant region comprising (i) any one of SEQ ID NOs: 8 and 14; and (ii) SEQ ID NO: 9. In some embodiments, an anti-IL-4Rα antibody or an antigen-binding portion thereof provided herein may comprise the six CDR amino acid sequences of any one of the clones in Table 1 or 2 and any one of the Fc region amino acid sequences in Table 5. In some embodiments, an anti-IL-4Rα antibody or an antigen-binding portion thereof provided herein may comprise an immunoglobulin heavy chain constant region comprising any one of the Fc region amino acid sequences in Table 5 and an immunoglobulin light chain constant region that is a kappa light chain constant region or a lambda light chain constant region.

In some embodiments, provided herein is an anti-IL-4Rα antibody or an antigen-binding portion thereof, wherein the antibody comprises a VH region, a VL region, a heavy chain constant region, and a light chain constant region, wherein (a) the VH region amino acid sequence comprises a HCDR1 comprising SEQ ID NO: 1, a HCDR2 comprising SEQ ID NO: 2 and a HCDR3 comprising SEQ ID NO: 3; the VL region amino acid sequence comprises a LCDR1 comprising SEQ ID NO: 4, a LCDR2 comprising LGS and a LCDR3 comprising SEQ ID NO: 5; and the heavy chain constant region comprises any one of SEQ ID NOS: 8 and 14 or (b) the VH region amino acid sequence comprises a HCDR1 comprising SEQ ID NO: 1, a HCDR2 comprising SEQ ID NO: 10 and a HCDR3 comprising SEQ ID NO: 11; the VL region amino acid sequence comprises a LCDR1 comprising SEQ ID NO: 4, a LCDR2 comprising LGS and a LCDR3 comprising SEQ ID NO: 5; the heavy chain constant region comprises any one of SEQ ID NOS: 8 and 14; and the light chain constant region comprises SEQ ID NO: 9.

In some embodiments, provided herein is an anti-IL-4Rα antibody or an antigen-binding portion thereof, wherein the antibody or antigen-binding portion comprises a VH region and a VL region, wherein (a) the VH region amino acid sequence comprises or consists of SEQ ID NO: 6; and the VL region amino acid sequence comprises or consists of SEQ ID NO: 7; or (b) the VH region amino acid sequence comprises or consists of SEQ ID NO: 12; and the VL region amino acid sequence comprises or consists of SEQ ID NO: 13.

In some embodiments, provided herein is an anti-IL-4Rα antibody or an antigen-binding portion thereof, wherein the antibody or antigen-binding portion comprises a VH region, a VL region, a heavy chain constant region, and a light chain constant region, wherein (a) the VH region amino acid sequence comprises or consists of SEQ ID NO: 6; the VL region amino acid sequence comprises or consists of SEQ ID NO: 7; the heavy chain constant region comprises or consists of SEQ ID NO: 8; and the light chain constant region comprises or consists of SEQ ID NO: 9; or (b) the VH region amino acid sequence comprises or consists of SEQ ID NO: 12; the VL region amino acid sequence comprises or consists of SEQ ID NO: 13; the heavy chain constant region comprises or consists of SEQ ID NO: 14; and the light chain constant region comprises or consists of SEQ ID NO: 9.

In some embodiments, provided herein is an anti-IL-4Rα antibody or an antigen-binding portion thereof, wherein the antibody or antigen-binding portion binds specifically to human IL-4Rα and (a) antagonizes proliferation of human IL-4Rα+ cells, such as TF-1 cells, when stimulated by human IL-4 and/or IL-13; and/or (b) inhibits secretion of one or more cytokines associated with allergic inflammation (e.g., TARC).

In some embodiments, an anti-IL-4Rα antibody or antigen-binding portion provided herein has one or more desirable pharmacokinetic properties. In some embodiments, an anti-IL-4Rα antibody or antigen-binding portion provided herein exhibits one or more improved pharmacokinetic properties compared to an anti-IL-4Rα antibody comprising an HCDR1 of GFTFRDYA (SEQ ID NO: 1), HCDR2 of ISGSGGNT (SEQ ID NO: 10), HCDR3 of AKDRLSITIRPRYYGLDV (SEQ ID NO: 15), LCDR1 of QSLLYSIGYNY (SEQ ID NO: 4), LCDR2 of LGS, and LCDR3 of MQALQTPYT (SEQ ID NO: 5). In some embodiments, an anti-IL-4Rα antibody or antigen-binding portion provided herein exhibits one or more improved pharmacokinetic properties compared to an anti-IL-4Rα antibody comprising a VH region of SEQ ID NO: 16 and a VL region of SEQ ID NO: 7.

In some embodiments, an anti-IL-4Rα antibody or an antigen-binding portion has increased binding affinity for a human recycling receptor. In some embodiments, an anti-IL-4Rα antibody or an antigen-binding portion has increased binding affinity for a human FcγRn receptor. In some embodiments, the increased affinity is at pH 6.0. In some embodiments, binding affinity of an anti-IL-4Rα antibody or an antigen-binding portion thereof for a human FcγRn receptor may be measured by BIACORE® analysis (e.g., by Biacore® 8K using a CM5 chip) and may be represented by K_{D}. In some embodiments, binding affinity of an anti-IL-4Rα antibody or an antigen-binding portion thereof for a human FcγRn receptor may be increased by about 5 to about 20 fold. In some embodiments, binding affinity of an anti-IL-4Rα antibody or an antigen-binding portion thereof for a human FcγRn receptor may be increased by at least 5, at least 6, at least 7, at least 8, at least 9, at least 10, at least 11, at least 12, at least 13, at least 14, at least 15, at least 16, at least 17, at least 18, at least 19 or at least 20 fold.

In some embodiments, an anti-IL-4Rα antibody or an antigen-binding portion has at least one improved pharmacokinetic property compared to dupilumab. In some embodiments, the one or more pharmacokinetic properties are determined by predictive software. In some embodiments, the predictive software is PK-Sim and/or MOBI. In some embodiments, the one or more pharmacokinetic properties comprise half-life, Cmax, clearance (CL), volume of distribution (V_{d}), volume of distribution at steady state (Vₛₛ), and/or area under the curve (AUC)_{0-∞} or (AUC)₀₋ₜ.

In some embodiments, an anti-IL-4Rα antibody or antigen-binding portion provided herein has reduced immunogenicity. In some embodiments, an anti-IL-4Rα antibody or antigen-binding portion exhibits immunogenicity that is substantially similar to that of an anti-IL-4Rα antibody comprising an HCDR1 of SEQ ID NO: 1, HCDR2 of SEQ ID NO: 10, HCDR3 of SEQ ID NO: 15, LCDR1 of SEQ ID NO: 4, LCDR2 of LGS and LCDR3 of SEQ ID NO: 5. In some embodiments, an anti-IL-4Rα antibody or antigen-binding portion exhibits immunogenicity that is substantially similar to that of an anti-IL-4Rα antibody comprising a VH region of SEQ ID NO: 16 and a VL region of SEQ ID NO: 7. As used herein, “substantially similar immunogenicity” refers to an immunogenicity value that is below 200% of that of the comparator antibody. In some embodiments, an anti-IL-4Rα antibody or antigen-binding portion exhibits reduced immunogenicity compared to an anti-IL-13 antibody comprising HCDR1 of AYSVN (SEQ ID NO: 17), HCDR2 of MIWGDGKIVYNSALKS (SEQ ID NO: 18), HCDR3 of DGYYPYAMDN (SEQ ID NO: 19), LCDR1 of RASKSVDSYGNSFMH (SEQ ID NO: 20), LCDR2 of LASNLES (SEQ ID NO: 21) and LCDR3 of QQNNEDPRT (SEQ ID NO: 22). In some embodiments, an anti-IL-4Rα antibody or antigen-binding portion exhibits immunogenicity that is substantially similar to that of an anti-IL13 antibody comprising a VH region of SEQ ID NO: 23; and a VL region of SEQ ID NO: 24.

In some examples, the reduced immunogenicity is predicted in silico or is measured in vitro, in vivo, or ex vivo. In some embodiments, immunogenicity is predicted in silico by AlphaMHC software (WeComput Technology). In some embodiments, the predictive software provides an immunogenicity score. In some embodiments, the immunogenicity score of the anti-IL-4Rα antibody or an antigen-binding portion is increased by no more than 5%, no more than 10%, no more than 15%, no more than 20%, no more than 25%, no more than 30%, no more than 35%, no more than 40%, no more than 45%, no more than 50%, no more than 60%, no more than 70%, no more than 80%, no more than 90%, no more than 100%, no more than 120%, no more than 140%, no more than 160%, no more than 180%, or no more than 200%, compared to an anti-IL-4Rα antibody comprising an HCDR1 of SEQ ID NO: 1, HCDR2 of SEQ ID NO: 10, HCDR3 of SEQ ID NO: 15, LCDR1 of SEQ ID NO: 4, LCDR2 of LGS and LCDR3 of SEQ ID NO: 5. In some embodiments, the immunogenicity score of the anti-IL-4Rα antibody or an antigen-binding portion is increased by no more than 5%, compared to an anti-IL-4Rα antibody comprising an HCDR1 of SEQ ID NO: 1, HCDR2 of SEQ ID NO: 10, HCDR3 of SEQ ID NO: 15, LCDR1 of SEQ ID NO: 4, LCDR2 of LGS and LCDR3 of SEQ ID NO: 5. In some embodiments, the immunogenicity score of the anti-IL-4Rα antibody or an antigen-binding portion is increased by no more than 30%, compared to an anti-IL-4Rα antibody comprising an HCDR1 of SEQ ID NO: 1, HCDR2 of SEQ ID NO: 10, HCDR3 of SEQ ID NO: 15, LCDR1 of SEQ ID NO: 4, LCDR2 of LGS and LCDR3 of SEQ ID NO: 5.

In some embodiments, the immunogenicity is measured via cytokine release from cells (e.g., whole blood or PBMCs from healthy donors) contacted with the anti-IL-4Rα antibody or antigen-binding portion thereof. In some embodiments, immunogenicity by cytokine release from cells is increased by no more than 20%, no more than 30%, no more than 40%, no more than 50%, no more than 60%, no more than 70%, no more than 80%, no more than 90%, no more than 100%, no more than 120%, no more than 140%, no more than 160%, no more than 180%, no more than 200%, no more than 250%, or no more than 300%, compared to an anti-IL-4Rα antibody comprising a VH region amino acid sequence comprising an HCDR1 of SEQ ID NO: 1, HCDR2 of SEQ ID NO: 10, HCDR3 of SEQ ID NO: 15, LCDR1 of SEQ ID NO: 4, LCDR2 of LGS and LCDR3 of SEQ ID NO: 5.

In some embodiments, an IL-4Rα antibody or an antigen-binding portion thereof may comprise a human IgG4 constant region comprising the amino acid substitution S228P and a kappa light chain constant region.

Further provided herein is an immunoconjugate comprising an anti-IL-4Rα antibody or an antigen-binding portion thereof, linked to a therapeutic agent. In some embodiments, the therapeutic agent is an immunomodulatory agent or a therapeutic nucleic acid.

Examples of suitable therapeutic agents include, but are not limited to, immunomodulatory agents. Further therapeutic agents include a therapeutic nucleic acid, such as a gene encoding an immunomodulatory agent. These drug descriptors are not mutually exclusive, and thus a therapeutic agent may be described using one or more of the above terms.

### Pharmaceutical Compositions

The anti-IL-4Rα antibodies and antigen-binding portions provided herein (also referred to herein as “active compounds”) can be incorporated into pharmaceutical compositions suitable for administration. Such compositions typically comprise an anti-IL-4Rα antibody or antigen-binding portion (or an immunoconjugate comprising said antibody or portion), and a pharmaceutically acceptable carrier, diluent or excipient. Such materials should be non-toxic and should not interfere with the efficacy of the anti-IL-4Rα antibody or antigen-binding fragment thereof. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the anti-IL-4Rα antibody or antigen-binding portion thereof, an increase in its lifespan and/or in its efficacy in the body or an increase in its solubility in solution.

Provided herein is a pharmaceutical composition comprising (i) an anti-IL-4Rα antibody or an antigen-binding portion thereof, wherein the antibody or antigen-binding portion comprises a VH region and a VL region, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5; and (ii) a pharmaceutically acceptable carrier, diluent or excipient; or (b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5.

Provided herein is a pharmaceutical composition comprising (i) an anti-IL-4Rα antibody or an antigen-binding portion thereof, wherein the antibody or antigen-binding portion comprises a VH region and a VL region, wherein: (a) the VH region amino acid sequence comprises or consists of SEQ ID NO: 6 and the VL region amino acid sequence comprises or consists of SEQ ID NO: 7; or (b) the VH region amino acid sequence comprises or consists of SEQ ID NO: 12 and the VL region amino acid sequence comprises or consists of SEQ ID NO: 13; and (ii) a pharmaceutically acceptable carrier, diluent or excipient.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the anti-IL-4Rα antibody or antigen-binding portion thereof may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Antibodies

Provided herein is a nucleic acid molecule (e.g., an isolated nucleic acid molecule) encoding an amino acid sequence of an anti-IL-4Rα antibody or anti-IL-4Rα antigen-binding portion described herein (or an amino acid sequence of a (i) VH region, (ii) a VL region, or (iii) both a VH region and a VL region of an antibody or antigen-binding portion). Further provided herein is a nucleic acid molecule (e.g., an isolated nucleic acid molecule) encoding (i) a heavy chain, (ii) a light chain, or (iii) both a heavy chain and a light chain of an anti-IL-4Rα antibody or anti-IL-4Rα antigen-binding portion described herein. In some embodiments, a nucleic acid molecule encoding a VH region, a VL region, a heavy chain or a light chain comprises a signal sequence. In some embodiments, a nucleic acid molecule encoding a VH region, a VL region, a heavy chain or a light chain does not comprise a signal sequence.

In some embodiments, a nucleic acid molecule encodes an amino acid sequence of a VH region and a VL region of an anti-IL-4Rα antibody or an antigen-binding portion thereof, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5; or (b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5. In some embodiments, a nucleic acid molecule further encodes a human framework region amino acid sequence.

In some embodiments, a nucleic acid molecule encodes an amino acid sequence of a VH region and a VL region of an anti-IL-4Rα antibody or an antigen-binding portion thereof, wherein (a) the VH region amino acid sequence comprises or consists of SEQ ID NO: 6 and the VL region amino acid sequence comprises or consists of SEQ ID NO: 7; or (b) the VH region amino acid sequence comprises or consists of SEQ ID NO: 12 and the VL region amino acid sequence comprises or consists of SEQ ID NO: 13.

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a VH region and a VL region of an anti-IL-4Rα antibody or an antigen-binding portion thereof. In some embodiments, a recombinant host cell comprises (i) a first vector or a first nucleic acid molecule encoding a VH region of an anti-IL-4Rα antibody or an antigen-binding portion thereof and (ii) a second vector or a second nucleic acid molecule encoding a VL region of an anti-IL-4Rα antibody or an antigen-binding portion thereof.

Antibody molecules of the invention, or antigen-binding portion thereof, can be produced using techniques well known in the art, for example, recombinant technologies, phage display technologies, synthetic technologies, computational technologies or combinations of such technologies or other technologies readily known in the art.

Further provided herein is a method for producing an anti-IL-4Rα antibody or an antigen-binding portion thereof, the method comprising: culturing a recombinant host cell comprising an expression vector described herein under conditions whereby the nucleic acid segment is expressed, thereby producing the anti-IL-4Rα antibody or antigen-binding portion. The antibody or antigen-binding portion may then be isolated from the host cell or culture. Anti-IL-4Rα antibodies and antigen-binding portions thereof can be produced by any of a variety of methods known to those skilled in the art. In certain embodiments, anti-IL-4Rα antibodies and antigen-binding portions thereof can be produced recombinantly. For example, nucleic acid sequences encoding one or more of SEQ ID NOs: 1-19, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, antibody light chain proteins and heavy chain proteins are produced in a cell with a signal sequence that is removed upon production of a mature anti-IL-4Rα antibody or antigen-binding portion thereof.

Those skilled in the art will be able to determine whether an antibody or antigen-binding portion comprising a given polypeptide sequence binds to IL-4Rα protein without undue experimentation using standard methodologies, for example, Western blots, ELISA, and the like.

### Uses of Antibodies

Provided herein are methods and uses of the anti-IL-4Rα antibodies, anti-IL-4Rα antigen-binding portions, immunoconjugates and pharmaceutical compositions described herein for providing a therapeutic benefit to a subject with an immune-mediated disease or disorder.

Provided herein is a method for treating or preventing a disease or condition in a subject, comprising administering to the subject a therapeutically effective amount of an antibody, antigen-binding portion, immunoconjugate or pharmaceutical composition disclosed herein. In some embodiments, the antibody or an antigen-binding portion thereof comprises a VH region and a VL region, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5; or (b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5. In some embodiments, the method comprises administering to the subject a therapeutically effective amount of an anti-IL-4Rα antibody or an antigen-binding portion thereof, wherein the antibody or antigen-binding portion comprises a VH region and a VL region, wherein: (a) the VH region amino acid sequence comprises or consists of SEQ ID NO: 6 and the VL region amino acid sequence comprises or consists of SEQ ID NO: 7; or (b) the VH region amino acid sequence comprises or consists of SEQ ID NO: 12 and the VL region amino acid sequence comprises or consists of SEQ ID NO: 13.

Provided herein is a method for reducing or improving at least one symptom of a disease or condition in a subject, the method comprising administering to the subject a therapeutically effective amount of an antibody, antigen-binding portion, immunoconjugate or pharmaceutical composition disclosed herein. In some embodiments, the antibody or an antigen-binding portion thereof comprises a VH region and a VL region, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5; or (b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5. In some embodiments, the method comprises administering to the subject a therapeutically effective amount of an anti-IL-4Rα antibody or an antigen-binding portion thereof, wherein the antibody or antigen-binding portion comprises a VH region and a VL region, wherein: (a) the VH region amino acid sequence comprises or consists of SEQ ID NO: 6 and the VL region amino acid sequence comprises or consists of SEQ ID NO: 7; or (b) the VH region amino acid sequence comprises or consists of SEQ ID NO: 12 and the VL region amino acid sequence comprises or consists of SEQ ID NO: 13.

In some embodiments, the method results in improvement of one or more pharmacokinetic properties. In some embodiments, the method results in an improvement in immunogenicity. In some embodiments, the method results in reduced IL-4/IL-13 signaling. In some embodiments, the disease or condition is associated with increased IL-4/IL-13 production.

In some embodiments, the disease or condition is atopic dermatitis. In some embodiments, the disease or condition is chronic obstructive pulmonary disorder (COPD), asthma, prurigo nodularis, or chronic rhinosinusitis with nasal polyps (CRSwNP).

Provided herein is method for reducing IL-4/IL-13 signalling, wherein the method comprises contacting a cell with an antibody, antigen-binding portion, immunoconjugate or pharmaceutical composition disclosed herein. In some embodiments, the antibody or an antigen-binding portion thereof comprises a VH region and a VL region, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5; or (b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5. In some embodiments, the method comprises administering to the subject a therapeutically effective amount of an anti-IL-4Rα antibody or an antigen-binding portion thereof, wherein the antibody or antigen-binding portion comprises a VH region and a VL region, wherein: (a) the VH region amino acid sequence comprises or consists of SEQ ID NO: 6 and the VL region amino acid sequence comprises or consists of SEQ ID NO: 7; or (b) the VH region amino acid sequence comprises or consists of SEQ ID NO: 12 and the VL region amino acid sequence comprises or consists of SEQ ID NO: 13.

In some embodiments, the method results in reduced proliferation of the cell. In some embodiments, the method results in reduced production of a cytokine. In some embodiments, the cytokine is thymus- and activation-regulated chemokine (TARC). In some embodiments, the cytokine is CCL33 (MDC).

As used herein, the term “effective amount” or “therapeutically effective amount” refers to the amount of a pharmaceutical agent, e.g., an anti-IL-4Rα antibody or an antigen-binding portion thereof, which is sufficient to reduce or ameliorate the severity and/or duration of a disease, e.g., atopic dermatitis, chronic obstructive pulmonary disorder (COPD), asthma, prurigo nodularis, or chronic rhinosinusitis with nasal polyps (CRSwNP), or one or more symptoms thereof, prevent the advancement of a disease, cause regression of a disease, prevent the recurrence, development, onset or progression of one or more symptoms associated with a disease, detect a disease, or enhance or improve the prophylactic or therapeutic effect(s) of another related therapy (e.g., prophylactic or therapeutic agent) for a IL-4Rα-mediated disease.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are known in the art (Ledermann J. A. et al., 1991, Int. J. Cancer 47:659-664; Bagshawe K. D. et al., 1991, Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

In some embodiments, anti-IL-4Rα antibody molecules and antigen-binding portions as described herein may be administered as sub-cutaneous injections. Sub-cutaneous injections may be administered using an auto-injector, for example for long term prophylaxis/treatment.

In some embodiments, the therapeutic effect of an anti-IL-4Rα antibody or an antigen-binding portion thereof may persist for several half-lives, depending on the dose. For example, the therapeutic effect of a single dose of an anti-IL-4Rα antibody or an antigen-binding portion thereof may persist in a subject for 1 month or more, 2 months or more, 3 months or more, 4 months or more, 5 months or more, or 6 months or more.

In some embodiments, a subject may be treated with an anti-IL-4Rα antibody or an anti-IL-4Rα antigen-binding portion, an immunoconjugate or a pharmaceutical composition described herein and an additional therapeutic agent or therapy that is used to treat a IL-4Rα-mediated disease or disorder or a symptom or complication of a IL-4Rα-mediated disease or disorder. The anti-IL-4Rα antibody or an anti-IL-4Rα antigen-binding portion and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Further provided herein is an anti-IL-4Rα antibody or an anti-IL-4Rα antigen-binding portion, an immunoconjugate or a pharmaceutical composition described herein, for use in the treatment of a disease or a disorder.

Provided herein is an anti-IL-4Rα antibody or an anti-IL-4Rα antigen-binding portion, an immunoconjugate or a pharmaceutical composition described herein, for use as a medicament.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular systems biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

“Antibody-drug conjugate” and “immunoconjugate” refer to an antibody molecule, or antigen-binding portion thereof, including antibody derivatives, that binds to IL-4Rα and is conjugated to cytotoxic, cytostatic and/or therapeutic agents.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example, nuclear magnetic resonance spectroscopy).

The term “potency” is a measurement of biological activity and may be designated as IC₅₀, EC₅₀, or effective concentration of an antibody or antibody drug conjugate to the antigen IL-4Rα to inhibit 50% of activity measured in a IL-4Rα activity assay as described herein.

The term “inhibit” or “neutralize” as used herein with respect to bioactivity of an antibody disclosed herein means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to IL-4Rα.

In the present description, any concentration range, percentage range, ratio range, or integer range is to be understood to include the value of any integer within the recited range and, when appropriate, fractions thereof (such as one tenth and one hundredth of an integer), unless otherwise indicated. The use of the alternative (e.g., “or”) should be understood to mean either one, both, or any combination thereof of the alternatives. As used herein, the terms “include” and “comprise” are used synonymously.

The section headings used herein are for organizational purposes only and are not to be construed as limiting the subject matter described.

All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Any of the aspects and embodiments described herein can be combined with any other aspect or embodiment as disclosed here in the Summary, in the Drawings, and/or in the Detailed Description, including the below specific, non-limiting, examples/embodiments of the present disclosure.

TABLE 1
Annotated amino acid sequences of antibody IL-4Rα-02-0084

| Domain or Region | Sequence | SEQ ID NO |
|---|---|---|
| Heavy chain variable | EVQLVESGGGLEQPGGSLRLSCAGSGFTFRDYAMTWVRQAPG | 6 |
| region | KGLEWVSSISGSGGRTYYADSVKGRFTISRDNSKNTLYLQMN | |
| | SLRAEDTAVYYCAKDRLSITIRPRYYGLEVWGQGTTVTVSS | |
| Heavy chain CDR1 | GFTFRDYA | 1 |
| Heavy chain CDR2 | ISGSGGRT | 2 |
| Heavy chain CDR3 | AKDRLSITIRPRYYGLEV | 3 |
| Light chain variable | DIVMTQSPLSLPVTPGEPASISCRSSQSLLYSIGYNYLDWYLQK | 7 |
| region | SGQSPQLLIYLGSNRASGVPDRFSGSGSGTDFTLKISRVEAEDV | |
| | GFYYCMQALQTPYTFGQGTKLEIK | |
| Light chain CDR1 | QSLLYSIGYNY | 4 |
| Light chain CDR2 | LGS | N/A |
| Light chain CDR3 | MQALQTPYT | 5 |

CDR sequences are underlined in variable region sequences.

TABLE 2
Annotated amino acid sequences of antibody IL-4Rα-02-011

| Domain or Region | Sequence | SEQ ID NO |
|---|---|---|
| Heavy chain variable | EVQLVESGGGLVQPGGSLRLSCAGSGFTFRDYAMTWVRQAP | 12 |
| region | GKGLEWVSSISGSGGNTYYADSVKGRFTISRDNSKNTLYLQM | |
| | NSLRAEDTAVYYCAKDRLSITIRPRYYGLWVWGQGTTVTVSS | |
| Heavy chain CDR1 | GFTFRDYA | 1 |
| Heavy chain CDR2 | ISGSGGNT | 10 |
| Heavy chain CDR3 | AKDRLSITIRPRYYGLWV | 11 |
| Light chain variable | DIVMTQSPLSLPVTPGEPASISCRSSQSLLYSIGYNYLDWYLQK | 13 |
| region | SGQSPKLLIYLGSNRASGVPDRFSGSGSGTDFTLKISRVEAEDV | |
| | GFYYCMQALQTPYTFGQGTKLEIK | |
| Light chain CDR1 | QSLLYSIGYNY | 4 |
| Light chain CDR2 | LGS | N/A |
| Light chain CDR3 | MQALQTPYT | 5 |

CDR sequences are underlined in variable region sequences.

TABLE 3
Exemplary antibody constant region amino acid sequences

| Domain or Region | Sequence | SEQ ID NO |
|---|---|---|
| IL-4Rα-02-0084 | ASTKGPSVFPLAPSSKSTSGGTAALGCLVKDYFPEPVTVSWNS | 8 |
| heavy chain constant | GALTSGVHTFPAVLQSSGLYSLSSVVTVPSSSLGTQTYICNVN | |
| region | HKPSNTKVDKKVEPKSCDKTHTCPPCPAPEAAGGPSVFLFPPK | |
| | PKDTLYITREPEVTCVVVDVSHEDPEVKFNWYVDGVEVHNA | |
| | KTKPREEQYNSTYRVVSVLTVLHQDWLNGKEYKCKVSNKALPA | |
| | PIEKTISKAKGQPREPQVYTLPPSRDELTKNQVSLTCLVKGF | |
| | YPSDIAVEWESNGQPENNYKTTPPVLDSDGSFFLYSKLTVDKS | |
| | RWQQGNVFSCSVMHEALHNHYTQKSLSLSPG | |
| IL-4Rα-02-011 heavy | ASTKGPSVFPLAPSSKSTSGGTAALGCLVKDYFPEPVTVSWNS | 14 |
| chain constant region | GALTSGVHTFPAVLQSSGLYSLSSVVTVPSSSLGTQTYICNVN | |
| | HKPSNTKVDKKVEPKSCDKTHTCPPCPAPEAAGGPSVFLFPPK | |
| | PKDTLYITREPEVTCVVVDVSHEDPEVKFNWYVDGVEVHNA | |
| | KTKPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDKS | |
| | RWQQGNVFSCSVMHEALHNHYTQKSLSLSPG | |
| Light chain constant | RTVAAPSVFIFPPSDEQLKSGTASVVCLLNNFYPREAKVQWKV | 9 |
| region | DNALQSGNSQESVTEQDSKDSTYSLSSTLTLSKADYEKHKVY | |
| | ACEVTHQGLSSPVTKSFNRGEC | |

TABLE 4
Annotated amino acid sequences of dupilumab

| Domain or Region | Sequence | SEQ ID NO |
|---|---|---|
| Heavy chain variable | EVQLVESGGGLEQPGGSLRLSCAGSGFTFRDYAMTWVRQAPG | 16 |
| region | KGLEWVSSISGSGGNTYYADSVKGRFTISRDNSKNTLYLQMN | |
| | SLRAEDTAVYYCAKDRLSITIRPRYYGLDVWGQGTTVTVSS | |
| Heavy chain CDR1 | GFTFRDYA | 1 |
| Heavy chain CDR2 | ISGSGGNT | 10 |
| Heavy chain CDR3 | AKDRLSITIRPRYYGLDV | 15 |
| Light chain variable | DIVMTQSPLSLPVTPGEPASISCRSSQSLLYSIGYNYLDWYLQK | 7 |
| region | SGQSPQLLIYLGSNRASGVPDRFSGSGSGTDFTLKISRVEAEDV | |
| | GFYYCMQALQTPYTFGQGTKLEIK | |
| Light chain CDR1 | QSLLYSIGYNY | 4 |
| Light chain CDR2 | LGS | N/A |
| Light chain CDR3 | MQALQTPYT | 5 |

CDR sequences are underlined in variable region sequences.

TABLE 5
Annotated amino acid sequences of lebrikizumab

| Domain or Region | Sequence | SEQ ID NO |
|---|---|---|
| Heavy chain variable | QVTLRESGPAL VKPTQTLTLTCTVSGFSLSAYSVNWIRQPPGK | 23 |
| region | ALEWLAMIWGDGKIVYNSALKSRLTISKDTSKNQVVLTMTN | |
| | MDPVDTATYYCAGDGYYPYAMDNWGQGSLVTVSS | |
| Heavy chain CDR1 | AYSVN | 17 |
| Heavy chain CDR2 | MIWGDGKIVYNSALKS | 18 |
| Heavy chain CDR3 | DGYYPYAMDN | 19 |
| Light chain variable | DIVMTQSPDSLSVSLGERATINCRASKSVDSYGNSFMHWYQQ | 24 |
| region | KPGQPPKLLIYLASNLESGVPDRFSGSGSGTDFTLTISSLQA | |
| | EDVAVYYCQQNNEDPRTFGGGTKVEIK | |
| Light chain CDR1 | RASKSVDSYGNSFMH | 20 |
| Light chain CDR2 | LASNLES | 21 |
| Light chain CDR3 | QQNNEDPRT | 22 |

CDR sequences are underlined in variable region sequences.

TABLE 6
Exemplary IL-4Rα sequences

| Domain or Region | Sequence | SEQ ID NO |
|---|---|---|
| Human IL-4Rα | MGWLCSGLLFPVSCLVLLQVASSGNMKVLQEPTCVSDYMSIS | 25 |
| | TCEWKMNGPTNCSTELRLLYQLVFLLSEAHTCIPENNGGAGC | |
| | VCHLLMDDVVSADNYTLDLWAGQQLLWKGSFKPSEHVKPR | |
| | APGNLTVHTNVSDTLLLTWSNPYPPDNYLYNHLTYAVNIWSE | |
| | NDPADFRIYNVTYLEPSLRIAASTLKSGISYRARVRAWAQCYN | |
| | TTWSEWSPSTKWHNSYREPFEQHLLLGVSVSCIVILAVCLLCY | |
| | VSITKIKKEWWDQIPNPARSRLVAIIIQDAQGSQWEKRSRGQEP | |
| | AKCPHWKNCLTKLLPCFLEHNMKRDEDPHKAAKEMPFQGSG | |
| | KSAWCPVEISKTVLWPESISVVRCVELFEAPVECEEEEEVEEEK | |
| | GSFCASPESSRDDFQEGREGIVARLTESLFLDLLGEENGGFCQQ | |
| | DMGESCLLPPSGSTSAHMPWDEFPSAGPKEAPPWGKEQPLHL | |
| | EPSPPASPTQSPDNLTCTETPLVIAGNPAYRSFSNSLSQSPCPRE | |
| | LGPDPLLARHLEEVEPEMPCVPQLSEPTTVPQPEPETWEQILRR | |
| | NVLQHGAAAAPVSAPTSGYQEFVHAVEQGGTQASAVVGLGP | |
| | PGEAGYKAFSSLLASSAVSPEKCGFGASSGEEGYKPFQDLIPG | |
| | CPGDPAPVPVPLFTFGLDREPPRSPQSSHLPSSSPEHLGLEPGEK | |
| | VEDMPKPPLPQEQATDPLVDSLGSGIVYSALTCHLCGHLKQC | |
| | HGQEDGGQTPVMASPCCGCCCGDRSSPPTTPLRAPDPSPGGVP | |
| | LEASLCPASLAPSGISEKSKSSSSFHPAPGNAQSSSQTPKIVNFV | |
| | SVGPTYMRVS | |
| Cynomolgus | MGWLCSGLLFPVSCLVLLQVASSGSMKVLQEPACVSDYMSIS | 26 |
| macaque IL-4Rα | TCEWKMGGPTNCSAELRLLYQLVFQSSETHTCVPENNGGVGC | |
| | VCHLLMDDVVSMDNYTLDLWAGQQLLWKGSFKPSEHVKPR | |
| | APGNLTVHTNVSDTVLLTWSNPYPPDNYLYNDLTYAVNIWSE | |
| | NDPAYSRIHNVTYLKPTLRIPASTLKSGISYRARVRAWAQHYN | |
| | TTWSEWSPSTKWYNSYREPFEQRLLWGVSAACVFILFFCLSCY | |
| | FSVTKIKKEWWDQIPNPARSHLVAIIIQDAQESQWEKRSRGQE | |
| | AAKCPYWKNCLTKLLPCFLEHNMKRDEDPHKAVKDLPFRGS | |
| | GKSAWCPVEISKTVLWPESISVVRCVELFEAPVECKEEEEVEE | |
| | EKGSFCTSSESNRDDFQEGREGIVARLTESLFLDLLGGENGGFF | |
| | QQDMGESCLLPPLGSTSAHVPWDEFPSAGPKEVPPWGKEQPL | |
| | HQEPSPPASPTQSPDNPTCTEMPLVISSNPAYRSFSNSLSQSPCP | |
| | RELGPDPLLARHLEEVDPEMPCAPQLSEPTTVAPAEPETWEQI | |
| | LRRNVLQHGAAAAPASAPTSGYREFVHAVQQGGIQASAVAG | |
| | LGPPGEAGYKAFSSLLASSAVSPGECGFGASSGEEGYKPFQDL | |
| | TPGCPGDPAPVPVPLFTFGLDREPPHSPQSSHLPSNSPEHLALEP | |
| | GEKVEDMQKPPLPPEQATDPLGDNLGSGIVYSALTCHLCGHL | |
| | KQCHGQEDGGQAPVVASPCCGCCCGDRSSPPTTPLRAPDPSL | |
| | GGVPLEASLCLASLAPSGISEKSKSSLSFHPAPGSAQSSSQTPQI | |
| | VNFVSVGPTCMRVS | |
| Rhesus monkey IL- | MGWLCSGLLFPVSCLVLLQVASSGSMKVLQEPTCVSDYMSIS | 27 |
| 4Rα | TCEWKMGGPTNCSAELRLLYQLVFQSSETHTCVPENNGGVGC | |
| | VCHLLMDDVVSMDNYTLDLWAGQQLLWKGSFKPSEHVKPR | |
| | APGNLTVHTNVSDTVLLTWSNPYPPDNYLYNDLTYAVNIWSE | |
| | NDLADSRIHNVTYLKPTLRIPASTLKSGISYRARVRAWAQHYN | |
| | TTWSEWSPSTKWYNSYREPFEQRLLWGVSAACVFILFFCLSCY | |
| | FSVTKIKKEWWDQIPNPARSHLVAIIIQDAQESQWEKRSRGQE | |
| | AAKCPYWKNCLTKLLPCFLEHNMKRDEDPHKAVKDLPFRGS | |
| | GKSAWCPVEISKTVLWPESISVVRCVELFEAPVECKEEEEVEE | |
| | EKGSFCTSSESNRDDFQEGREGIVARLTESLFLDLLGGENGGFF | |
| | QQDMGESCLLPPLGSTSAHVPWDEFPSAGPKEVPPWGKEQPL | |
| | HQEPSPPASPTQSPDNPTCTEMPLVISSNPAYRSFSNSLSQSPCP | |
| | RELGPDPLLARHLEEVDPEMPCDPQLSEPTTVAPAEPETWEQI | |
| | LRRNVLQHGAAAAPASAPTSGYREFVHAVQQGGIQASAVAG | |
| | LGPPGEAGYKAFSSLLASSAVSPGECGFGASSGEEGYKPFQDL | |
| | TPGCPGDPAPVPVPLFTFGLDREPPHSPQSSHLPSNSPEHLALEP | |
| | GEKVEDMQKPPLPPEQATDPLGDNLGSGIVYSALTCHLCGHL | |
| | KQCHGQEDGGQAPVVASPCCGCCCGDRSSPPTTPLRAPDPSL | |
| | GGVPLEASLCPASLAPSGISEKSKSSLSFHPAPGSAQSSSQTPQI | |
| | VNFVSVGPTCMRVS | |

### EXAMPLES

### Example 1. Anti-IL-4Rα Antibodies with Superior Inhibition of TF-1 Cell Proliferation

The ability of anti-IL-4Rα antibody candidates AB-IL-4Rα-02-0084 and AB-IL-4Rα-02-011 to block IL-4/IL-13 signaling was assessed in a TF-1 cell proliferation assay, in comparison to the benchmark IL-4 antibody dupilumab. Proliferation, which in this cell line occurs in response to IL-4 and/or IL-13-induced stimulation, was quantified by measuring absorbance at 450 nm using a colorimetric WST-8 assay, and the IC50 was calculated for each antibody.

Both AB-IL-4Rα-02-0084 and AB-IL-4Rα-02-011 demonstrated superior inhibition of TF-1 cell proliferation versus dupilumab, with IC50s of 0.256 and 0.242 nM, respectively, compared to 0.951 nM for dupilumab.

| Antibody | IC50 (nM) |
|---|---|
| Dupilumab | 0.951 |
| AB-IL-4Rα-02-0084 | 0.256 |
| AB-IL-4Rα-02-011 | 0.242 |

### Example 2. Anti-IL-4Rα Antibodies with Superior Inhibition of TARC Secretion

TARC (thymus and activation-regulated chemokine) is a chemokine whose production is induced by signaling through the IL-4Rα pathway. The ability of anti-IL4-Ra antibody candidates AB-IL-4Rα-02-0084 and AB-IL-4Rα-02-011 to effectively to block IL-4/IL-13 signaling was measured in a TARC secretion assay. Cells were incubated in the presence of IL-4 and a serial dilution of either AB-IL-4Rα-02-0084, AB-IL-4Rα-02-011, or dupilumab. TARC concentration in cell supernatant was quantified by ELISA after 24 hours. Both AB-IL-4Rα-02-0084 and AB-IL-4Rα-02-011 showed markedly stronger inhibition of TARC secretion compared to dupilumab.

| Antibody | IC50 (nM) |
|---|---|
| Dupilumab | 5.118 |
| AB-IL-4Rα-02-0084 | 2.007 |
| AB-IL-4Rα-02-011 | 2.781 |

### Example 3. Anti-IL-4Rα Antibodies with Low Immunogenicity

Immunogenicity of anti-IL-4Rα antibody candidates AB-IL-4Rα-02-0084 and AB-IL-4Rα-02-011 was predicted using AlphaMHC (WeComput Technology). Both AB-IL-4Rα-02-0084 and AB-IL-4Rα-02-011 are predicted to have low immunogenicity, comparable to that of the standard of care antibody dupilumab.

| Antibody | Immunogenicity Score |
|---|---|
| Dupilumab | 0.29 |
| AB-IL-4Rα-02-0084 | 0.37 |
| AB-IL-4Rα-02-011 | 0.28 |

Immunogenicity of anti-IL4-Ra antibody candidates AB-IL-4Rα-02-0084 and AB-IL-4Rα-02-011 is tested by cytokine release assay in PBMCs and/or whole blood from healthy donors. Either or both antibodies are expected to produce comparable or lower levels of pro-inflammatory cytokines compared to dupilumab.

### Example 4. Anti-IL-4Rα Antibodies with Reduced FcRn Affinity

Binding affinity of anti-IL-4Rα antibody candidates AB-IL-4Rα-02-0084 and AB-IL-4Rα-02-011 for FcRn is measured by SPR compared to dupilumab. FcRn binding kinetics at pH 6.0 and pH 7.4 are measured using a Biacore® 8K instrument (Cytiva) using CM5 chip at pH. Both AB-IL-4Rα-02-0084 and AB-IL-4Rα-02-011 are predicted to have reduced binding affinity to FcRn compared to dupilumab. Without wishing to be bound by theory, the reduced FcRn affinity of these antibodies may be due to mutations to the immunoglobulin heavy chain region such as those shown in FIG. 1.

### Example 5. Anti-IL-4Rα Antibodies with Improved Pharmacokinetic Properties

Pharmacokinetics of anti-IL-4Rα antibody candidates AB-IL-4Rα-02-0084 and AB-IL-4Rα-02-011 is predicted using PK-Sim and MOBI, which are physiologically based pharmacokinetic (PBPK) modeling software, based on clinical data from ongoing trials. This in silico approach allows determination of pharmacokinetics (PK) and translation of in vitro data for first-in-human (FIH) starting dose selection.

1. An anti-interleukin-4 (IL4) receptor alpha (IL-4Rα) antibody or an antigen-binding portion thereof, wherein the antibody or antigen-binding portion comprises a heavy chain variable (VH) region and a light chain variable (VL) region wherein:
(a) the VH region amino acid sequence comprises a heavy chain complementarity determining region 1 (HCDR1) comprising the amino acid sequence of SEQ ID NO: 1, a heavy chain complementarity determining region 2 (HCDR2) comprising the amino acid sequence of SEQ ID NO: 2 and a heavy chain complementarity determining region 3 (HCDR3) comprising the amino acid sequence of SEQ ID NO: 3; and the VL region amino acid sequence comprises a light chain complementarity determining region 1 (LCDR1) comprising the amino acid sequence of SEQ ID NO: 4, a light chain complementarity determining region 2 (LCDR2) comprising the amino acid sequence of LGS and a light chain complementarity determining region 1 (LCDR3) comprising the amino acid sequence of SEQ ID NO: 5; or
(b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5.

2. The antibody or antigen-binding portion of claim 1, wherein:
(a) the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 6 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 7; or
(b) the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 12 and the VL region amino acid sequence has at least 90%, %, at least 91%, at least 92%, at least 93%, at least 94%, at least 95%, at least 96%, at least 97%, at least 98%, or at least 99% sequence identity to the amino acid sequence of SEQ ID NO: 13.

3. The antibody or antigen-binding portion of claim 1, wherein:
(a) the VH region comprises the amino acid sequence of SEQ ID NO: 6 and the VL region comprises the amino acid sequence of SEQ ID NO: 7; or
(b) the VH region comprises the amino acid sequence of SEQ ID NO: 12 and the VL region comprises the amino acid sequence of SEQ ID NO: 13.

4. The antibody or antigen-binding portion of claim 1, wherein the antibody or antigen-binding portion is humanized or chimeric.

5. The antibody or antigen-binding portion of claim 1, wherein the VH region, the VL region, or both the VH and the VL region comprise one or more human framework region amino acid sequences.

6. The antibody or antigen-binding portion of claim 1, wherein the antibody or antigen-binding portion comprises an immunoglobulin constant region.

7. The antibody or antigen-binding portion of claim 6, wherein the immunoglobulin constant region is IgG, IgE, IgM, IgD, IgA or IgY.

8. The antibody or antigen-binding portion of claim 7, wherein the immunoglobulin constant region is IgG1, IgG2, IgG3, IgG4, IgA1 or IgA2.

9. (canceled)

10. The antibody or antigen-binding portion of claim 6, wherein the immunoglobulin constant region is a human IgG1 or IgG4 constant region comprising the amino acid substitutions L234A and L235A, wherein numbering is according to the EU index as in Kabat.

11. The antibody or antigen-binding portion of claim 6, wherein the immunoglobulin constant region is a human IgG1 or IgG4 constant region comprising the amino acid substitutions M252Y, S254T, and T256E, wherein numbering is according to the EU index as in Kabat.

12. The antibody or antigen-binding portion of claim 8, wherein the immunoglobulin constant region comprises (i) any one of SEQ ID NOs: 8 or 14, and (ii) SEQ ID NO: 9.

13. (canceled)

14. The antibody or antigen-binding portion of claim 1, wherein the antibody or antigen-binding portion is an Fab, an Fab′, an F(ab′)₂, an Fv, an scFv, a maxibody, a minibody, a diabody, a triabody, a tetrabody, or a bis-scFv.

15. The antibody or antigen-binding portion of claim 1, wherein the antibody or antigen-binding portion is bispecific.

16. An immunoconjugate comprising the antibody or antigen-binding portion of claim 1, linked to a therapeutic agent.

17. A pharmaceutical composition comprising an anti-interleukin-4 (IL4) receptor alpha (IL-4Rα) antibody or an antigen-binding portion thereof, wherein the antibody or antigen-binding portion comprises a heavy chain variable (VH) region and a light chain variable (VL) region wherein:
(a) the VH region amino acid sequence comprises a heavy chain complementarity determining region 1 (HCDR1) comprising the amino acid sequence SEQ ID NO: 1, a heavy chain complementarity determining region 2 (HCDR2) comprising the amino acid sequence of SEQ ID NO: 2 and a heavy chain complementarity determining region 3 (HCDR3) comprising the amino acid sequence of SEQ ID NO: 3; and the VL region amino acid sequence comprises a light chain complementarity determining region 1 (LCDR1) comprising the amino acid sequence of SEQ ID NO: 4, a light chain complementarity determining region 2 (LCDR2) comprising the amino acid sequence of LGS and a light chain complementarity determining region 3 (LCDR3) comprising the amino acid sequence of SEQ ID NO: 5; or
(b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5; and
a pharmaceutically acceptable carrier, diluent or excipient.

18. (canceled)

19. (canceled)

20. (canceled)

21. (canceled)

22. (canceled)

23. A method for treating or preventing a disease or condition in a subject, comprising administering to the subject a therapeutically effective amount of an anti-interleukin-4 (IL4) receptor alpha (IL-4Rα) antibody or an antigen-binding portion thereof, wherein the antibody or antigen-binding portion comprises a heavy chain variable (VH) region and a light chain variable (VL) region wherein:
(a) the VH region amino acid sequence comprises a heavy chain complementarity determining region 1 (HCDR1) comprising the amino acid sequence of SEQ ID NO: 1, a heavy chain complementarity determining region 2 (HCDR2) comprising the amino acid sequence of SEQ ID NO: 2 and a heavy chain complementarity determining region 3 (HCDR3) comprising the amino acid sequence of SEQ ID NO: 3; and the VL region amino acid sequence comprises a light chain complementarity determining region 1 (LCDR1) comprising the amino acid sequence of SEQ ID NO: 4, a light chain complementarity determining region 2 (LCDR2) comprising the amino acid sequence of LGS and a light chain complementarity determining region 3 (LCDR3) comprising the amino acid sequence of SEQ ID NO: 5; or
(b) the VH region amino acid sequence comprises a HCDR1 comprising the amino acid sequence of SEQ ID NO: 1, a HCDR2 comprising the amino acid sequence of SEQ ID NO: 10 and a HCDR3 comprising the amino acid sequence of SEQ ID NO: 11; and the VL region amino acid sequence comprises a LCDR1 comprising the amino acid sequence of SEQ ID NO: 4, a LCDR2 comprising the amino acid sequence of LGS and a LCDR3 comprising the amino acid sequence of SEQ ID NO: 5.

24. (canceled)

25. (canceled)

26. (canceled)

27. (canceled)

28. (canceled)

29. The method of claim 23, wherein the disease or condition is atopic dermatitis.

30. The method of claim 23, wherein the disease or condition is chronic obstructive pulmonary disorder (COPD), asthma, prurigo nodularis, or chronic rhinosinusitis with nasal polyps (CRSwNP).

31. A method for reducing IL-4/IL-13 signalling, wherein the method comprises contacting a cell with the antibody or antigen-binding portion of claim 1.

32. (canceled)

33. (canceled)

34. The antibody or antigen-binding portion of claim 1, for use as a medicament.
